(19) United States
(12) Patent Application Publication
(10) Pub. No.: US 2023/0404429 A1
(43) Pub. Date: Dec. 21, 2023

(54) WHOLE BODY PLETHYSMOGRAPH SYSTEMS AND METHODS TO CONTROL THE GAS TIGHTNESS INSIDE A CABINET OF A WHOLE BODY PLETHYSMOGRAPH

(71) Applicant: VYAIRE MEDICAL GMBH, Höchberg (DE)

(72) Inventors: Michael HEINL, Höchberg (DE); Jürgen REINSTÄDTLER, Höchberg (DE)

(21) Appl. No.: 18/251,116

(22) PCT Filed: Oct. 30, 2020
(86) PCT No.: PCT/IB2020/060222

§ 371 Date: Apr. 28, 2023

(51) Int. Cl. A61B 5/08

(52) U.S. Cl. CPC A61B 5/0806

(57) ABSTRACT

A whole-body-plethysmograph 11 for examination of a lung function of subjects. The whole-body-plethysmograph has a cabinet 12 with a cabinet opening 13 and a cabinet closure 14 for gas-tight sealing of the cabinet opening 13. A pressure control device 15 is provided, and the pressure control device 15 is configured to provide a desired negative gas pressure inside the cabinet 12 to seal the cabinet opening 13 by the cabinet closure 14. Furthermore, methods to control the gas tightness inside a cabinet 12 of the whole-body-plethysmograph are disclosed.

The present invention relates to whole-body-plethysmograph systems and methods to control the gas tightness inside a cabinet of a whole-body-plethysmograph according to the independent claims.

A whole-body plethysmography itself is assumed to be known. Whole-body plethysmography is a method for determining bronchial flow resistance and thoracic gas volumes in practical clinical applications. The physical basis is Boyle-Mariotte's law derived from the general gas equation. For methodological reasons, the volume-constant whole-body plethysmographic method has prevailed over the pressure-constant system. The pressure changes in the cabinet are usually adiabatic and can be calculated according to the formulas for adiabatic pressure changes. Whole body plethysmographic measurements are based on a very sensitive pressure measurement of the changing internal pressure in the closed cabinet.

DE 10 2015 107 811 A1 discloses a whole-body-plethysmograph for examination of the lung function of subjects, comprising a cabinet with a cabinet opening and a cabinet closure for gas-tight sealing of the cabinet opening. Furthermore, said whole-body-plethysmograph does comprise a pressure control device and a control system, where said cabinet pressure is set stabile to provide a reliable measurement result during the examination of the lung function of subjects. The cabinet closure must comprise a reliable sealing system with locking system to provide a reliable measurement and to suppress leakage in the cabinet. Such a cabinet comprise a heavy magnetic lock or a heavy mechanic lock as well as a heavy and stiff cabinet wall material, which is heavy to transport. Especially, magnetic locks need a strong magnetic field during operation, which may influence the patient during the examination.

Aim of the invention is to avoid at least some of the drawbacks of the prior art and to improve a whole-body-plethysmograph, in particular, to provide a whole-body-plethysmograph system with an improved, simplified operational safety, while the influence of the patient during the examination of the lung function is reduced. Furthermore, methods to control the gas tightness inside a cabinet of a whole-body-plethysmograph system is provided, to improve and simplify the operational safety of said whole-body-plethysmograph systems.

At least some of said problems are accomplished by the whole-body-plethysmograph systems and methods to control the gas tightness inside a cabinet of said plethysmograph systems according to the independent claims.

The inventive whole-body-plethysmograph for examination of the lung function of subjects comprises a cabinet with a cabinet opening and a cabinet closure for gas-tight sealing of the cabinet opening. Furthermore, a pressure control device is provided, while said pressure control device is configured to provide a desired negative gas pressure inside said cabinet to seal said cabinet opening by said cabinet closure.

The advantage of the system is, that said cabinet or said cabinet closure does not need any locking system for sealing the cabinet during the examination of the lung function of the patient. Said cabinet closure of such a whole-body-plethysmograph, typically needs a stiff structure with reliable sealings around said cabinet opening. Said cabinet closure may be a door or a window, which is used for placing said subject/patient in said whole-body-plethysmograph. During the examination, a constant negative gas pressure is provided, to reliably lock the cabinet closure and seal the cabinet. The inventive whole-body-plethysmograph utilizes the force generated by the pressure difference between the ambient pressure and the pressure inside the cabinet, affecting the cabinet closure. Furthermore, using said whole-body-plethysmograph, a pressure measurement apparatus can be avoided for monitoring the sealing of the cabinet.

Said pressure control device comprises a valve or a flow control device for ventilating the inside of the cabinet, especially to reopen the cabinet closure for releasing the subject from the cabinet.

Preferably, the desired negative gas pressure inside the cabinet is between 0.3 and 2.0 kPa below the ambient pressure. A negative gas pressure in this range guarantees a sufficient sealing of the cabinet closure and will not influence the wellbeing of the subject during the examination. In addition, a negative gas pressure below 2.0 kPa will not influence the examination of the lung function of the patient.

Preferably, the desired negative gas pressure inside the cabinet is 0.5 kPa. The force applied to the cabinet closure depends on the area of the cabinet closure inside the cabinet. A negative gas pressure of 0.5 kPa sufficiently seals the cabinet closure for a cabinet opening for a human body and for sufficient sealing of the cabinet, where the time for providing the negative gas pressure is minimized.

Further preferably, the pressure control device is a pump, or a blower or a vacuum system, for providing a gas outlet flow from said cabinet to the ambient outside of the cabinet. Said gas outlet flow generates said negative gas pressure in said cabinet. Said pump may be used to quickly create the negative gas pressure in said cabinet. Said pressure control device comprises a valve or a flow control device for quickly ventilate ventilating the cabinet after examination of the lung function of the patient.

Said blower may be used as a pressure control device. In this case the blower itself may produce the negative gas pressure inside said cabinet during operation, and in addition, the blower may work as a valve for ventilating the cabinet, when the operation of the fan of the blower is stopped.

Said cabinet may be connected to a vacuum system in a hospital. Said vacuum system typically is part of a hospital, especially of a central vacuum supply in the hospital. Said vacuum system typically comprises a valve or a flow control, which is connected to the cabinet of said whole-body-plethysmograph.

Preferably a control system is provided, while said control system is configured to control the pressure control device. Said control system is electrical connected to the pressure control device and sends control data to said pressure control device. Said negative gas pressure is quickly and reliably set in said cabinet, using said pressure control device.

Further preferably, said control system is configured to stabilize the pressure condition between said gas outlet flow and a gas inlet flow by means of leakages of the cabinet. The examination of the lung function will cause a very small pressure variation inside the cabinet. Those pressure variations are detected during examination. Thus, the control system is configured that a slow pressure variation inside the cabinet may be suppressed and the cabinet disclosure is reliable sealed. Thus, the examination of the lung function of subjects is performed under stable conditions.

Alternatively, or in addition, said control system is configured to stabilize the pressure condition between said gas outlet flow and a gas inlet flow by means of warming of the gas inside the cabinet caused by the subject/patient. Said patient shows a natural body warming of the air inside the cabinet, which influences the pressure condition inside said cabinet, and therefore may falsify said examination of said lung function. Thus, the system, described herein, is used to perform the examination of the lung function of subjects under enhanced stable conditions.

Optionally at least a pressure sensor for detecting said negative gas pressure inside said cabinet is arranged in the cabinet and is connected to said control system for sending said measured gas pressure data to said control system. Said pressure sensor is used to monitor the negative gas pressure in said cabinet. Thus, a reproducible sealing of said cabinet closure is provided. Said pressure sensor is used to provide a feedback system to easily control the gas pressure condition inside the cabinet. Thus, a cabinet closure, made of a material with lower stiffness may be used, which reduces the costs of the whole system, described herein.

Further preferably, said control system comprises a first control program and a memory. Said first control program is configured to receive said detected pressure data from said gas pressure sensor and creates at least a control parameter to control the pressure control device. Said memory may comprise desired negative gas pressure data values, which are provided by the controller of the plethysmograph. In particular, said first control program is configured to perform a comparison of said desired negative gas pressure provided by said memory and a detected negative gas pressure inside said cabinet. Said control system is used to speed up the evacuation procedure before examination of the lung function of the patient. The desired pressure difference depends on the lower gas flow and the leakage of the cabinet. Advantageously, said control system is configured to control the pressure control device from a higher gas outlet flow to a lower gas outlet flow after a predefined waiting time.

Preferably, said control system is configured to control the pressure control device from a higher gas outlet flow to a lower gas outlet flow based on the above-mentioned comparison. Thus, the evacuation procedure before examination of the lung function of the patient is further speeded up and more patients may be examined per day. Thus, the overall cost for the examination is reduced significantly.

Further preferably, said control system comprises a further control program, which is configured to provide the relation of a gas outlet flow and at least a measured gas pressure data to provide an indication for the gas tightness of said cabinet closure. Thus, a feedback system could “remember” the previous desired pressure and make equilibration faster. This will additionally speed up the whole procedure before examination.

Preferably, at least a flow sensor for detecting the gas outlet flow in said cabinet is arranged in the cabinet and is connected to said control system for sending said measured gas outlet flow data to said control system. Measuring the gas flow out of the cabinet provides flow data, which may be used to control the system reproduceably.

Alternative to the flow sensor, the pump property or the blower property is, that the flow is determined by the speed of the pump or by the control voltage of the blower using said control system. Thus, the outlet flow may be easily be determined. In particular, measuring the speed of the pump provides information data, which may be used to control the system reproduceably. Furthermore, when utilizing a vacuum system, the flows can be determined by flow resistive elements or the flow rate of the valves used to control the gas flow.

Further preferably, said cabinet comprises a cabinet wall and said cabinet wall comprises a pressure dependent material, while said pressure dependent material changes its stiffness depending on the negative gas pressure inside said cabinet. Thus, small pressure changes inside the cabinet do not change the volume inside said cabinet, when the wall comprises a sufficient stiff wall. The cabinet wall becomes hard enough for small pressure changes inside the cabinet. Furthermore, the cabinet may be produced with less material and the cabinet may be transported easier, because it loses weight

Preferably, said cabinet closure is made of said dependent material, while said pressure dependent material changes its stiffness depending on the negative gas pressure inside said cabinet. The cabinet closure is lightweight and therefore the handling of the cabinet closure is easier and sustainable.

Further preferably, said pressure dependent material is arranged with a skeleton. The skeleton may be arranged inside or outside the pressure dependent material and supports the whole structure of the plethysmograph, when the system is not in operation. The pressure dependent material is preferably a plastic foil.

Preferably said pressure control device is arranged in said cabinet wall. Thus, the installation of said plethysmograph is easier. In particular said pressure control device is arranged in the floor of the cabinet.

Furthermore, the invention comprises a method to control the gas tightness inside a cabinet of a whole-body-plethysmograph, comprising a cabinet with a cabinet opening, a cabinet closure for gas-tight sealing of the cabinet opening, preferably whole-body-plethysmographs as mentioned herein, wherein said method comprising the steps:
a) Providing a gas outlet flow out of the cabinet with a pressure control device;
b) Providing a desired negative gas pressure inside said cabinet to lock said cabinet opening by said cabinet closure.

The advantage of said system is, that said cabinet or said cabinet closure does not need any locking system for sealing the cabinet during the examination of the lung function of the patient.

Preferably, a control system is provided and after step b), the gas outlet flow is controlled from a higher gas outlet flow to a lower gas outlet flow. The sealing of the cabinet is controlled reliably and reproducibly. The control of the outlet flow may start after a predefined waiting time.

Further preferably, said desired negative gas pressure is measured and is stored in a memory. The stored data may be used to speed up the system. Preferably said desired negative gas pressure is compared to an actual gas pressure in said cabinet to provide a quick feedback system. Therefore, more patients may be examined per day. Thus, the overall cost for the examination is reduced significantly.

Preferably, after step b), the pressure condition between said gas outlet flow and a gas inlet flow is stabilized based on leakages and/or warming of the gas inside the cabinet by the subject. Thus, a feedback system could “remember” the previous desired pressure and make equilibration faster.

Further preferably, after step b), an indication for the gas tightness of said cabinet closure is provided, which is preferably based on the relation of the gas outlet flow and at least a measured pressure data, preferably measured with a pressure sensor or a flow sensor.

Preferably, the stiffness of at least one wall of said cabinet is changed, preferably of said cabinet closure, based on said negative gas pressure inside the cabinet. The cabinet may be produced with less material and the cabinet may be transported and installed easier, because it loses weight compared to the state of the art whole-body-plethysmograph.

Furthermore, the invention comprises a whole-body-plethysmograph for examination of the lung function of subjects, comprising a cabinet with a cabinet opening, a cabinet closure for gas-tight sealing of the cabinet opening. Said cabinet comprises at least one cabinet wall, which is made of a pressure dependent material, while said pressure dependent material changes its stiffness depending on the gas pressure inside said cabinet.

Such a whole-body-plethysmograph may be operated with a positive gas pressure or a negative gas pressure, without using a separate locking system. Those whole-body-plethysmographs may be transported and installed for a first time in a first hospital place and if necessary, may be quickly re-installed at another place, e.g. patient's room, at another time. A whole-body-plethysmograph operating with a negative gas pressure is described in detail above. A whole-body-plethysmograph operating with a positive gas pressure comprises at least a cabinet closure, which is arranged inside the cabinet. This cabinet closure is pressed against the cabinet wall caused by the overpressure in the cabinet during operation. A skeleton for stabilization of the cabinet is not needed and the cabinet may be easily transported and installed. Alternatively, the cabinet closure may comprise a flexible zipper. Preferably, said cabinet closure is made of said dependent material.

Further preferably, a pressure control device is provided, while said pressure control device is configured to provide a desired negative or positive gas pressure inside said cabinet. The stiffness of the cabinet wall and/or the cabinet closure is reproduceable controlled and thus the operation conditions can be easily held stable during the examination. Preferably, a pressure control device is an external vacuum system or an external compressor. The overpressure must achieve a range that stabilizes the entire soft pressure dependent material and increases the stiffness of the material. Thus, small pressure changes inside the cabinet do not change the volume inside said cabinet. The wall becomes hard enough for small pressure changes inside the cabinet. Since no skeleton is necessary, this cabinet may be produced much smaller and lighter, especially when not in operation. Even a transportable version would be possible. The pressure source would probably be an external compressor or compressed air from the hospital wall. The gas flow can be adjusted by flow restrictors and valves. Since the pressure difference between the supply (compressor or wall connection) and the inside of the cabinet is large, the flow does not change when the pressure inside the cabinet changes a little.

Further preferred embodiments of said whole-body-plethysmograph are disclosed in the said whole-body-plethysmograph embodiments above.

Furthermore, the invention comprises a method to control the gas tightness and/or stiffness of a cabinet of a whole-body-plethysmograph, comprising a cabinet with a cabinet opening, a cabinet closure for gas-tight sealing of the cabinet opening, preferably whole-body-plethysmographs as mentioned, wherein said method comprising the steps:
a) Providing a gas outlet flow out of the cabinet or a gas inlet flow into the cabinet with a pressure control device;
b) Providing a desired gas pressure inside said cabinet to provide a desired stiffness of at least one cabinet wall or cabinet closure.

The advantage of said system is, that said cabinet or said cabinet closure does not need any locking system for sealing the cabinet during the examination of the lung function of the patient. Such a whole-body-plethysmograph may be operated with a positive gas pressure or a negative gas pressure. Those whole-body-plethysmographs may be transported and installed for a first time in a first hospital place and if necessary, may be quickly re-installed at another place, e.g. patient's room, at another time.

Further preferred steps to control the gas tightness inside a cabinet are disclosed in the said method above.

Further advantageous aspects of the invention are explained in the following by means of exemplary embodiments and the figures. In the drawings, it is shown in a schematic manner. Furthermore, a numeric counting within this application is just used to differ between said parts of said whole-body-plethysmograph.

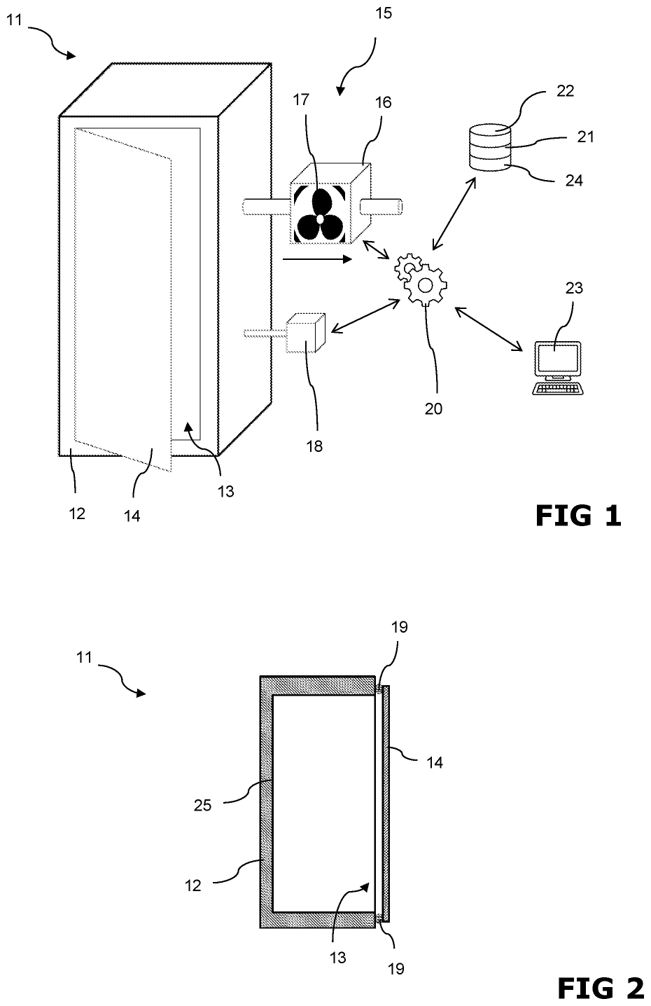

FIG. 1: A first embodiment of a whole-body-plethysmograph in an exploded-view,

FIG. 2: the whole-body-plethysmograph of FIG. 1 in a sectional view,

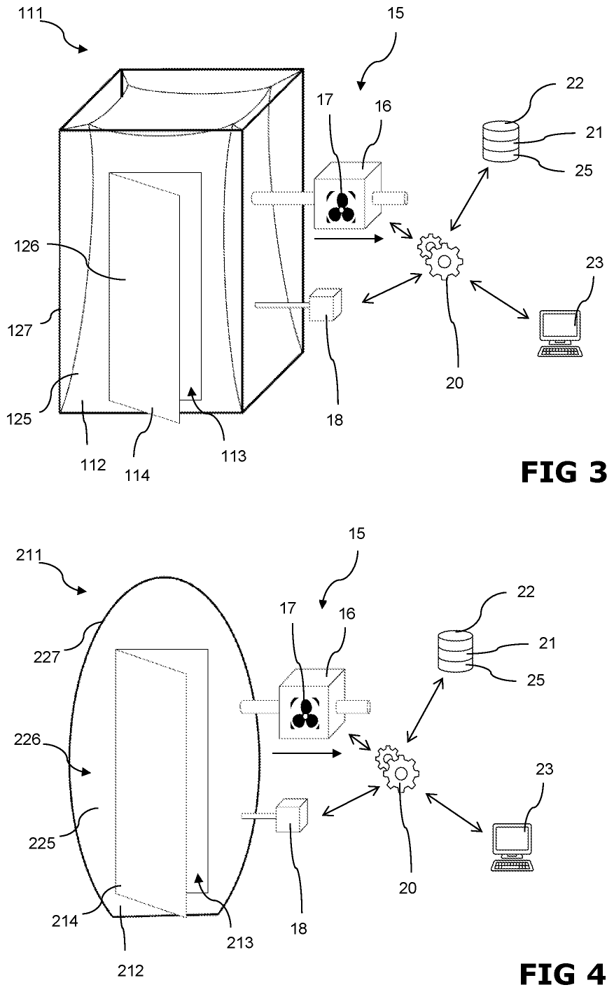

FIG. 3: A second embodiment of a whole-body-plethysmograph in an exploded-view,

FIG. 4: A third embodiment of a whole-body-plethysmograph in an exemplary-view, and

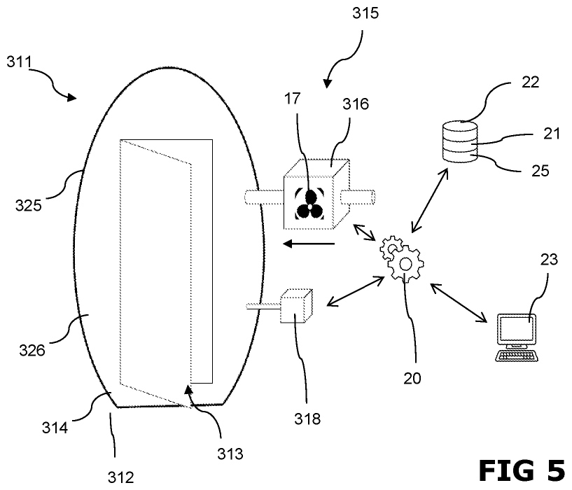

FIG. 5: A fourth embodiment of a whole-body-plethysmograph in an exemplary-view.

FIG. 1 and FIG. 2 discloses a first embodiment of a whole-body-plethysmograph 11 for examination of the lung function of subjects. The whole-body-plethysmograph 11 comprises a cabinet 12 with a cabinet wall 25, a cabinet opening 13 and a cabinet closure 14 for gas-tight sealing of the cabinet opening 13. During examination of the subject, which may be human or an animal, inside the cabinet 12, the cabinet closure 14, e.g. the door, must be closed and sealed against the ambient conditions outside the cabinet 12. Between said cabinet opening 13 and cabinet closure 14 a sealing 19 is arranged. The whole-body-plethysmograph 11 is connected to a pressure control device 15, which is configured to provide a desired negative gas pressure inside said cabinet 12 to seal said cabinet opening 13 by said cabinet closure 14. During the examination, a constant negative gas pressure of 0.5 kPa is provided, to reliably lock the cabinet closure 14 and seal the cabinet 12. The whole-body-plethysmograph utilizes the force generated by the pressure difference between the ambient pressure and the pressure inside the cabinet 12, affecting the cabinet closure 14. The disclosed pressure control device is a blower 16 with a fan 17. The blower 16 works as a valve for ventilating the cabinet 12, when the operation of the fan 17 of the blower 16 is stopped or the rotation speed of the fan 17 is reduced.

The blower 16 is connected to a control system 20, while said control system 20 is configured to control the blower 17. The control system 20 is configured to stabilize the pressure condition between said gas outlet flow and a gas inlet flow by means of leakages of the cabinet 12 and/or by means of warming of the gas inside the cabinet 12 caused by the subject/patient. The cabinet 12 comprises a pressure sensor 18 for detecting said negative gas pressure inside said cabinet 12 and is connected to said control system 20 for sending said measured gas pressure data to said control system 20. Said pressure sensor 18 is used to monitor the negative gas pressure in said cabinet 12. Said pressure sensor 18 is used to provide a feedback system to control the fan 17 and therefore the gas pressure condition inside the cabinet 12.

The control system 20 comprises a first control program 21 and a memory 22. Said first control program 21 is configured to receive said detected pressure data from said gas pressure sensor 18 and creates at least one control parameter to control the blower 16. Said memory 22 comprises desired negative gas pressure data values, which are provided by the controller 23 of the plethysmograph. The controller 23 is a computing unit, in particular with an input unit and a monitor. Said first control program 21 is configured to perform a comparison of said desired negative gas pressure provided by said memory 22 and a detected negative gas pressure inside said cabinet 12. The desired pressure difference depends on the gas flow and the leakage of the cabinet 12. The control system 20 is configured to control the blower 17 from a higher gas outlet flow to a lower gas outlet flow based on the above-mentioned comparison.

Furthermore, the control system 20 comprises a further control program 24, which is configured to provide the relation of a gas outlet flow and at least a measured gas pressure data to provide an indication for the gas tightness of said cabinet closure 14.

Alternatively, the pressure control device 15 comprises a pump or a vacuum system of the hospital, for providing a gas outlet flow from said cabinet 12 to the ambient outside of the cabinet 12 and comprises a valve or a flow control device for quickly ventilate the cabinet 12 after examination of the lung function of the patient. The flow outlet is e.g. determined by the speed of the pump.

FIG. 3 discloses a second embodiment of the a whole-body-plethysmograph 111 for examination of the lung function of subjects. The whole-body-plethysmograph 111 comprises a cabinet 112 with a cabinet opening 113 and a cabinet closure 114 for gas-tight sealing of the cabinet opening 112. During examination of the subject, the cabinet closure 114, e.g. the door, must be closed and sealed against the ambient conditions outside the cabinet 112. The whole-body-plethysmograph 111 is connected to a pressure control device 15, which is described in the embodiments disclosed above in FIG. 1 and FIG. 2.

Said cabinet 111 comprises a cabinet wall 125 and a skeleton 127. Said cabinet wall 125 comprises a pressure depended material 126, e.g. a plastic foil, while said pressure dependent material 126 changes its stiffness depending on the negative gas pressure inside said cabinet 112. Thus, small pressure changes inside the cabinet 112 do not change the gas volume inside said cabinet 112, when the cabinet wall 125 comprises a sufficiently stiff cabinet wall 125. At least said cabinet closure 114 is made of said dependent material 126. Such a cabinet closure 114 may be used in the a whole-body-plethysmograph 11, disclosed in FIG. 1 and FIG. 2, too. The skeleton 127 is arranged outside the pressure depended material 126 and is connected to the cabinet walls 125 and supports the whole structure of the plethysmograph 111, when the system is not in operation.

FIG. 4 discloses a third embodiment of the a whole-body-plethysmograph 211 for examination of the lung function of subjects. The plethysmograph 211 comprises a cabinet 212 with a cabinet opening 213 and a cabinet closure 214 for gas-tight sealing of the cabinet opening 212. During examination of the subject, the cabinet closure 214, e.g. the door, must be closed and sealed against the ambient conditions outside the cabinet 212. The whole-body-plethysmograph 211 is connected to a pressure control device 15, which is described in the embodiments disclosed above in FIG. 1 and FIG. 2. Said cabinet comprises at least one cabinet wall 225, which is made of a pressure dependent material 226, while said pressure dependent material 226 changes its stiffness depending on the gas pressure inside said cabinet 212. Such a whole-body-plethysmograph operates with a positive gas pressure or a negative gas pressure, without using a separate locking system.

Said cabinet 211 comprises a cabinet wall 225 and a skeleton 227. Said cabinet wall 225 comprises a pressure depended material 226, like a plastic foil, as described in the above in FIG. 3. The skeleton 227 is arranged inside the pressure depended material 226 and inside to the cabinet walls 225.

FIG. 5 discloses a fourth embodiment of the a whole-body-plethysmograph 311 for examination of the lung function of subjects. The whole-body-plethysmograph 311 comprises a cabinet 312 with a cabinet opening 313 and a cabinet closure 314 for gas-tight sealing of the cabinet opening 312. During examination of the subject, the cabinet closure 314, e.g. a flexible zipper door, must be closed and sealed against the ambient conditions outside the cabinet 312.

The whole-body-plethysmograph 311 is operating with a positive gas pressure. Therefore, the cabinet 312 comprises at least a cabinet closure 314, which is arranged inside the cabinet 312. This cabinet closure 314 is pressed against the cabinet wall 325 caused by the overpressure in the cabinet 312 during operation. A skeleton is not needed.

The whole-body-plethysmograph 311 is connected to a pressure control device 315, which is in general configured like the pressure control device disclosed above in FIG. 1 and FIG. 2. The pressure control device 315 is configured to provide a desired positive gas pressure (instead of a negative gas pressure) inside said cabinet 331. The stiffness of the cabinet wall 325 and/or the cabinet closure 314 is reproduceable controlled and thus the operation conditions can be easily held stable during the examination. The pressure control device is an external compressor 316 for providing a gas inlet flow. The gas flow sensor 318 detects the inlet flow into the cabinet 312. A gas pressures sensor detects the positive pressure in the cabinet 312. The overpressure must achieve a range that stabilizes the entire soft pressure dependent material 326 and increases the stiffness of the material.

A method to control the gas tightness inside a cabinet, e.g. for the cabinet of the whole-body-plethysmograph 11 of the FIG. 1 and FIG. 2. The following method may also be used in for the whole-body-plethysmograph 111 or 211 in FIG. 3 or FIG. 4. The method comprising the steps:
a) Providing a gas outlet flow out of the cabinet 12 with a pressure control device 15;
b) Providing a desired negative gas pressure inside said cabinet 12 to seal said cabinet opening 13 by said cabinet closure 14.

After step a), the gas outlet flow is controlled from a higher gas outlet flow to a lower gas outlet flow with the control system 20 and the stiffness of said cabinet closure 14 is controlled, based on said negative gas pressure inside the cabinet 12.

After step b), the pressure condition between said gas outlet flow and a gas inlet flow is stabilized based on leakages and/or warming of the gas inside the cabinet 12 by the subject.

During the method, a desired negative gas pressure is measured and is stored in a memory 22. The stored data may be used to speed up the system. The desired negative gas pressure is compared to an actual gas pressure, measured by the gas pressure sensor 18 in said cabinet 12 to provide a quick feedback system.

Further preferably, after step b), an indication for the gas tightness of said cabinet closure is provided, which is preferably based on the relation of the gas outlet flow and at least a measured pressure data, preferably measured with a pressure sensor or a flow sensor.

Another method to control the gas tightness inside a cabinet, e.g. for the cabinet of the whole-body-plethysmograph 211 of the FIG. 4. The following method may also be used in for the whole-body-plethysmograph 11, 211 in in FIG. 1, FIG. 2 or FIG. 3. The method comprising the steps:
a) Providing a gas outlet flow out of the cabinet 212 with a pressure control device 15;
b) Providing a desired gas negative pressure inside said cabinet 212 to provide a desired stiffness of at least one cabinet wall 226 or cabinet closure 214.

The advantage of said system is, that said cabinet 212 or said cabinet closure 214 does not need any locking system for sealing the cabinet 212 during the examination of the lung function of the patient.

Further preferred steps to control the gas tightness inside a cabinet 212 are disclosed in the said method above.

Another method to control the gas tightness inside a cabinet, e.g. for the cabinet 312 of the whole-body-plethysmograph 311 of the FIG. 5. The method comprising the steps:
a) Providing a gas inlet flow into the cabinet with a pressure control device 315;
b) Providing a desired positive gas pressure inside said cabinet 312 to provide a desired stiffness of at least one cabinet 312 wall or cabinet closure 314.

During the method, a desired positive gas pressure is measured and is stored in a memory 22. The stored data may be used to speed up the system. The desired positive gas pressure is compared to an actual gas pressure, measured by the gas pressure sensor or determined based on the measured data of the flow sensor 318 to provide a quick feedback system.

Further preferred steps to control the gas tightness inside a cabinet 312 are disclosed in the said methods above.

### LIST OF REFERENCES

11 whole-body-plethysmograph
12 Cabinet
13 cabinet opening
14 cabinet closure
15 pressure control device
16 Blower
17 Fan
18 gas pressure sensor
19 Sealing
20 control system
21 first control program
22 Memory
23 Controller
24 further control program
25 cabinet wall
111 whole-body-plethysmograph
112 Cabinet
113 cabinet opening
114 cabinet closure
125 cabinet wall
126 pressure depended material
127 Skeleton
211 whole-body-plethysmograph
212 Cabinet
213 cabinet opening
214 cabinet closure
225 cabinet wall
226 pressure depended material
227 Skeleton
311 whole-body-plethysmograph
312 Cabinet
313 cabinet opening
314 cabinet closure
315 pressure control device
316 external compressor
318 gas flow sensor
325 cabinet wall
326 pressure depended material

1-24. (canceled)

25. A whole-body-plethysmograph (11; 111; 211) for examination of a lung function of subjects, the plethysmograph comprising:
a cabinet (12; 112; 212) having a cabinet opening (13; 113; 213);
a cabinet closure (14; 114; 214) for gas-tight sealing of the cabinet opening (13; 113; 213);
a pressure control device (15) is provided, said pressure control device (15) is configured to provide a desired negative gas pressure inside said cabinet (12; 112; 212) to seal said cabinet opening (13; 113; 213) by said cabinet closure (14; 114; 214), while a pressure variation caused by the examination of the lung function is measured, and wherein the desired negative gas pressure inside the cabinet (12; 112; 212) is between 0.3 and 2.0 kPa below ambient pressure, preferably 0.5 kPa.

26. The plethysmograph according to claim 25, wherein the pressure control device (15) is a pump or a blower (16) or a vacuum system for providing a gas outlet flow from said cabinet (12; 112; 212) to the ambient outside the cabinet (12; 112; 212).

27. The plethysmograph according to claim 25, wherein a control system (20) is provided, said control system (20) is configured to control the pressure control device.

28. The plethysmograph according to claim 27, wherein said control system (20) is configured to stabilize a pressure condition between a gas outlet flow and a gas inlet flow by means of leakages and/or warming of gas inside the cabinet (12; 112; 212) by the subject.

29. The plethysmograph according to claim 27 wherein at least a pressure sensor (18) for detecting a negative gas pressure inside said cabinet (12; 112; 212) is arranged in the cabinet (12; 112; 212) and is connected to said control system (20) for sending measured pressure data to said control system (20).

30. The plethysmograph according to claim 29, wherein said control system (20) comprises a first control program and a memory (22), said first control program (21) is configured to perform a comparison of said desired negative gas pressure provided by said memory (22) and the detected negative gas pressure inside said cabinet (12; 112; 212).

31. The plethysmograph according to claim 30, wherein said control system (20) is configured to control the pressure control device (15) from a higher gas outlet flow to a lower gas outlet flow based on said comparison.

32. The plethysmograph according to claim 27, wherein said control system (20) comprises a further control program (24), which is configured to provide the relation of a gas outlet flow and at least a measured gas pressure data to provide an indication of gas tightness of said cabinet closure (14; 114; 214).

33. The plethysmograph according to claim 25, wherein at least a flow sensor for detecting a gas outlet flow in said cabinet (12; 112; 212) is arranged in the cabinet (12; 112; 212) and is connected to a control system (20) for sending measured gas outlet flow data to said control system (20).

34. The plethysmograph according to claim 25, wherein said cabinet (12; 112; 212) comprises a cabinet wall (25; 125; 225) and said cabinet wall (25; 125; 225) comprises a pressure dependent material (126; 226), said pressure dependent material (126; 226) changes stiffness depending on the negative gas pressure inside said cabinet (12; 112; 212).

35. The plethysmograph according to claim 34, wherein said pressure dependent material (126; 226), is arranged with a skeleton, and is preferably a plastic foil.

36. The plethysmograph according to claim 34, wherein said pressure control device (15) is arranged in said cabinet wall (25; 125; 225).

37. A method to control the gas tightness inside a cabinet of a whole-body-plethysmograph comprising a cabinet (12; 112; 212) with a cabinet opening, a cabinet closure (14; 114; 214) for gas-tight sealing of the cabinet opening, preferably a whole-body plethysmograph according to claim 25, wherein said method comprising steps of:
a) providing a gas outlet flow out of the cabinet (12; 112; 212) with a pressure control device (15);
b) providing a desired negative gas pressure inside said cabinet (12; 112; 212) to lock said cabinet opening (13; 113; 213) by said cabinet closure (14; 114; 214), while a pressure variation caused by the examination of the lung function is measured;
c) wherein the desired negative gas pressure inside the cabinet (12; 112; 212) is between 0.3 and 2.0 kPa below the ambient pressure, preferably kPa.

38. The method according to claim 37, wherein a control system (20) is provided and after step a), the gas outlet flow is controlled from a higher gas outlet flow to a lower gas outlet flow, preferably after a predefined waiting time.

39. The method according to claim 38, wherein said desired negative gas pressure is measured and is stored in a memory (22) and said desired negative gas pressure is compared to an actual gas pressure in said cabinet (12; 112; 212).

40. The method according to claim 37, further comprising after step b), stabilizing the pressure condition between said gas outlet flow and a gas inlet flow based on leakages and/or warming of the gas inside the cabinet (12; 112; 212) by the subject.

41. The method according to claim 37, further comprising after step b), providing an indication for the gas tightness of said cabinet closure (14; 114; 214), which is preferably based on the relation of the gas outlet flow and at least a measured pressure data, preferably measured with a pressure sensor (18) or a flow sensor or via a vacuum system.

42. The method according to claim 37, further comprising changing a stiffness of at least one wall of said cabinet (12; 112; 212), preferably of said cabinet closure (14; 114; 214), based on said negative gas pressure inside the cabinet (12; 112; 212).

43. A whole-body-plethysmograph for examination of a lung function of subjects, the whole-body-plethysmograph comprising a cabinet (12; 112; 212; 312) with a cabinet opening, a cabinet closure (14; 114; 214; 314) for gastight sealing of the cabinet opening, wherein said cabinet (12; 112; 212; 312) comprises at least one cabinet wall (25; 125; 225; 325), which is made of a pressure dependent material (126; 226; 326), and a stiffness of said pressure dependent material (126; 226; 326) changes depending on gas pressure inside said cabinet (12; 112; 212; 312).

44. The whole-body-plethysmograph according to claim 43, wherein said cabinet closure (14; 114; 214; 314) is made of said pressure dependent material (126; 226; 326).

45. The whole-body-plethysmograph according to claim 43, wherein a pressure control device (15; 315) is provided and said pressure control device (15; 315) is configured to provide a desired negative or positive gas pressure inside said cabinet (12; 112; 212; 312).

46. The whole-body-plethysmograph according to claim 45, wherein the pressure control device (15; 315) is an external vacuum system or an external compressor (316).

47. A method to control the gas tightness and/or stiffness of a cabinet of a whole-body-plethysmograph comprising a cabinet (12; 112; 212; 312) with a cabinet opening, a cabinet closure (14; 114; 214; 314) for gas-tight sealing of the cabinet opening, preferably a whole-body-plethysmograph according to claim 43, wherein said method comprising steps of:
a) providing a gas outlet flow out of the cabinet or a gas inlet flow into the cabinet (12; 112; 212; 312) with a pressure control device (15; 315);
b) providing a desired gas pressure inside said cabinet (12; 112; 212; 312) to provide a desired stiffness of at least one cabinet wall (25; 125; 225; 325) or cabinet closure (14; 114; 214; 314) made of a pressure dependent material (126; 226; 326).
